(19) United States
(12) Patent Application Publication
(10) Pub. No.: US 2016/0310259 A1
(43) Pub. Date: Oct. 27, 2016

(54) METHOD AND COMPUTING AND PRINTING UNIT FOR THE CREATION OF A STENT GRAFT

(71) Applicant: Siemens Aktiengesellschaft, Muenchen (DE)

(72) Inventors: Stefan ASSMANN, Erlangen (DE); Alexander GRAFENBERG, Effeltrich (DE); Jan HILLE, Nuernberg (DE); David-Amadeus KEIL, Bamberg (DE); Rainer KUTH, Hoechstadt (DE); Jochen RUSS, Gremsdorf (DE)

(21) Appl. No.: 15/132,409

(22) Filed: Apr. 19, 2016

(30) Foreign Application Priority Data
Apr. 24, 2015 (DE) 102015207596.6

(51) Int. Cl. A61F 2/07; B29C 67/00

(52) U.S. Cl. CPC A61F 2/07; B29C 67/0051; B29C 67/0088; B29L 2031/753

(57) ABSTRACT

A method is disclosed where at least one first 3D image data set is received via a first interface, the first 3D image data set including 3D images of a vascular segment of a patient at different time points. On the basis of the first 3D image data set, both a first hemodynamic parameter of the vascular segment and the spatial course of the vascular segment are then determined. This additional information enables the calculation of a digital 3D model of the stent graft on the basis of the first hemodynamic parameter of the vascular segment and on the basis of the spatial course of the vascular segment. Hence, the 3D model takes into account the first hemodynamic parameter and the spatial course so that a stent graft based on the digital 3D model is adapted individually to the anatomy of a patient via a 3D printer.

### PRIORITY STATEMENT

The present application hereby claims priority under 35 U.S.C. §119 to German patent application number DE 102015207596.6 filed Apr. 24, 2015, the entire contents of which are hereby incorporated herein by reference.

### FIELD

At least one embodiment of the invention generally relates to a method and/or a computing and printing unit for the creation of a stent graft.

### BACKGROUND

Blood vessels can change due to ageing, disease, stress or the consumption of alcohol, tobacco and other drugs. For example, vessels can constrict due to plaque deposition, wherein the risk of infarction is increased by the constriction. Furthermore, aneurysms and other dangerous changes to blood vessels can occur. Changes of this kind can be diagnosed by use of medical imaging such as computed tomography or magnetic resonance imaging. Various options are known for reducing the danger to the patient due to blood vessel changes. For example, a constricted blood vessel can be circumvented by a bypass or the constricted vessel can be dilated and stabilized by a stent graft, in particular by a so-called stent.

Conventional stent grafts are prefabricated and not individually adapted to a patient. New types of stent grafts for the aorta are based on a digital 3D model of the aorta and are produced via 3D printing. 3D models can furthermore be created on the basis of 3D image data. A novel stent graft of this type is described in the unexamined patent application US 2013 0296 998 A1. The stent graft described therein has openings for blood vessels branching-off from the aorta. This greatly restricts the possibilities of an individual adaptation of the stent graft.

### SUMMARY

At least one embodiment of the present invention provides a stent graft, which is individually adapted to the anatomy of a patient.

At least one embodiment of the present invention is directed to a method, a computing and printing unit, an imaging device, a computer program product, a computer-readable medium and/or a stent graft.

The following describes the embodiments according to the method of the invention. Any features, advantages or alternative embodiments can also be applied to the other claimed subject matter and vice versa. In other words, the substantive claims (which are, for example directed at an apparatus) can also be developed with the features described or claimed in connection with a method. Here, the corresponding functional features of the method are embodied by corresponding substantive modules.

At least one embodiment of the invention is directed to a method for creating a stent graft. In the method, at least one first 3D image data set is received via a first interface, wherein the first 3D image data set comprises 3D images of a vascular segment of a patient at different time points. The inventors have recognized that, on the basis of the first 3D image data set, both a first hemodynamic parameter of the vascular segment and the spatial course of the vascular segment can be determined via a determination unit. This additional information enables the calculation of a digital 3D model of the stent graft on the basis of the first hemodynamic parameter of the vascular segment and on the basis of the spatial course of the vascular segment via a computing unit. Therefore, the 3D model takes account of the first hemodynamic parameter and the spatial course such that a stent graft is adapted individually to the anatomy of a patient on the basis of the digital 3D model via a 3D printer. The stent graft is designed to support or replace the vascular segment.

Furthermore, at least one embodiment of the invention relates to a computing and printing unit for creating a stent graft, comprising the following units:

a first interface embodied to receive at least one first 3D image data set, wherein the first 3D image data set comprises 3D images of a vascular segment of a patient at different time points,

a determination unit embodied for the first determination of a first hemodynamic parameter of the vascular segment on the basis of the first image data set and the second determination of the spatial course of the vascular segment on the basis of the first 3D image data set,

a computing unit embodied to calculate a digital 3D model of the stent graft on the basis of the first hemodynamic parameter of the vascular segment and on the basis of the spatial course of the vascular segment,

a 3D printer embodied to create a stent graft on the basis of the digital 3D model.

A computing and printing unit of this kind can in particular be embodied to carry out the above-described method according to embodiments of the invention and its aspects. The computing and printing unit is embodied to carry out this method and its aspects in that the first interface, the determination unit, the computing unit and the 3D printer are embodied to carry out the corresponding method steps. At least one embodiment of the invention furthermore relates to an imaging device embodied to record the first 3D image data set comprising a computing and printing unit according to the invention.

At least one embodiment of the invention also relates to a computer program product with a computer program and a computer-readable medium. A substantially software-based implementation has the advantage that controller devices already used to date can be simply retrofitted with a software update in order to operate in accordance with at least one embodiment of the invention. In addition to the computer program, a computer program product of this kind can optionally comprise additional components such as, for example, documentation and/or additional components including hardware components, such as, for example, hardware keys (dongles etc.) for using the software.

### BRIEF DESCRIPTION OF THE DRAWINGS

The following describes and explains the invention in more detail with reference to the example embodiments shown in the figures, which show:

FIG. 1 a computing and printing unit,

FIG. 2 a network with a computing and printing unit,

FIG. 3 an imaging device,

FIG. 4 a 3D image of a vascular segment,

FIG. 5 a stent graft,

FIG. 6 a flow diagram of a method for the creation of a stent graft,

FIG. 7 a cross section through a stent graft.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

At least one embodiment of the invention is based on the fact that at least one first 3D image data set is received via a first interface, wherein the first 3D image data set comprises 3D images of a vascular segment of a patient at different time points. The inventors have recognized that, on the basis of the first 3D image data set, both a first hemodynamic parameter of the vascular segment and the spatial course of the vascular segment can be determined via a determination unit. This additional information enables the calculation of a digital 3D model of the stent graft on the basis of the first hemodynamic parameter of the vascular segment and on the basis of the spatial course of the vascular segment via a computing unit. Therefore, the 3D model takes account of the first hemodynamic parameter and the spatial course such that a stent graft is adapted individually to the anatomy of a patient on the basis of the digital 3D model via a 3D printer. The stent graft is designed to support or replace the vascular segment.

Hemodynamics describes the flow or movement in a vascular system and/or in a vascular segment, in particular as a function of physical parameters. Therefore, a hemodynamic parameter is a physical parameter suitable for describing the flow or movement of a fluid, in particular blood. If a hemodynamic parameter is taken into account during calculation of the 3D model, it is then possible to create a stent graft that is particularly reliable and particularly functional for a specific patient. In particular, it is possible for the 3D model to be calculated such that the stent graft together with the vascular segment and/or together with regions of the patient's vascular system adjacent to the vascular segment has a predetermined hemodynamic parameter or a predetermined value range for the hemodynamic parameter. A predetermined hemodynamic parameter of this kind or predetermined value range advantageously corresponds to a healthy patient. Therefore, the invention enables the production of a new generation of stent grafts, which are created such that the functional properties are individually adapted to the anatomy of the patient.

It is furthermore important to determine the spatial course of the vascular segment since this can differ greatly between different patients. If the 3D model is calculated on the basis of the course, the stent graft is individually adapted to the anatomy of the patient. The adaptation can be performed such that the stent graft has the shape of the vascular segment at least partially. In particular, this also enables stent grafts to be created for patients in whom a vascular segment has an unusual spatial course. The spatial course describes the 3D expansion of the vascular segment, that is in particular branches, contortions and further deviations from a straight course. It is important for the digital 3D model to be calculated on the basis of the spatial course to enable a fluid in the stent graft or in the vascular segment supported by the stent graft to flow in the predetermined way. This in particular enables the restoration of a flow such as that expected with a healthy patient. The fluid can, in particular, be blood.

Furthermore, it is possible to determine the first hemodynamic parameter taking account of the spatial course of the vascular segment. It is also possible to determine the first hemodynamic parameter by simulation. For example, it is possible to perform a flow simulation with which the flow of a fluid through the vascular segment or through the vascular system of the patient is simulated. The simulation can be based on the Navier-Stokes equation. Furthermore, the simulation can be performed numerically. Such a simulation can be based on as CFD (abbreviation for computational fluid dynamics). The simulation can be performed with FEM (abbreviation for finite element modeling).

With a simulation of this kind, some parameters (for example blood viscosity) are known and other parameters (such as the expansion of the vascular segment at different time points) are derived from the 3D image data set. It is now possible to derive a hemodynamic parameter therefrom, for example the average pressure during a cardiac cycle. A simulation of this kind enables the first hemodynamic parameter to be determined particularly precisely. It is also possible to calculate a plurality of different hemodynamic parameters depending upon which parameters of the simulation are known.

The stent graft is created via a 3D printer. A 3D printer of this kind creates the stent graft directly in accordance with the example of the 3D model, in particular by way of CAD (abbreviation for computer-aided design). Different materials can be used for the creation, in particular ceramics, metals, plastics and synthetic resins. At the same time, different materials can be connected to one another by a melting or adhesive process. Special variants of 3D printing include selective laser melting or electron beam melting for metals and selective laser sintering for plastics, ceramics and metals. Particularly advantageous for the present invention, is the (Poly)Jet method for plastics and synthetic resins. In this case, a plurality of layers of one or more plastics and/or synthetic resins is applied by one or more nozzle(s). Therefore, a 3D printer can comprise one or more nozzle(s) for the application of layers of plastic and/or synthetic resin.

The stent graft is made of biocompatible materials or coated therewith. Furthermore, the stent graft can have additional functions due to chemical treatment and/or treatment of the surface. For example, the stent graft can comprise pharmacologically active substances such that they are released into the patient's blood circulation.

The stent graft can have at least a compact conformation and an expanded conformation, wherein the compact conformation has a substantially smaller cross section than the expanded conformation. A substantially smaller cross section can in particular be achieved by reducing the cross-sectional area by more than 10%, 25% or 50%. In the compact conformation, the stent graft is designed to be transported through the patient's vascular system, in particular through an access to the vascular system in region of the groin, to the vascular segment. This makes the stent graft suitable for minimally invasive surgery. Furthermore, the stent graft is embodied to change from the compact conformation into the expanded conformation in the region of the vascular segment of the patient. In the expanded conformation, the stent graft performs its intended function and stabilizes the vascular segment in a particularly reliable way since the stent graft is individually adapted to the anatomy of the patient.

Alternatively, the stent graft can be unsuitable for minimally invasive surgery and only have a predetermined conformation. A stent graft with only a predetermined conformation is particularly simple to produce and can furthermore be embodied as particularly stable. A stent graft of this kind can nevertheless have an elasticity that effects a slight change to the predetermined conformation, for example due to the pressure of a fluid flowing through the stent graft. However, this does not substantially reduce or enlarge the cross section.

Furthermore, the vascular segment can be located in different regions of the body, in particular in the abdomen, in the extremities, or even in the brain. The vascular segment is suitable for transporting a fluid, in particular blood. The stent graft according to the invention is particularly suitable for supporting branched vascular segments. For example, the vascular segment can be a part of the aorta. This is because it is particularly important to provide a reliable stent graft with vascular segments of this kind. Furthermore, it is particularly advantageous to support branched vascular segments since branching varies very greatly between different patients. Therefore, it is also possible for the stent graft itself to be branched. Furthermore, it is very advantageous to provide a branched, coherent stent graft since, to date, it has only been possible to support branched vascular segments with a plurality of separate stent grafts. The method for using a plurality of separate stent grafts is complicated and hence prone to errors. Furthermore, a branched, coherent stent graft is more stable and better adapted to the anatomy of the patient than a plurality of separate stent grafts.

Therefore, the stent graft can also support a given vascular segment from the inside. Furthermore, however, the stent graft can also be embodied to replace a vascular segment. In the case of particularly greatly changed vascular segments, the replacement of this vascular segment can result in a more reliable function of the vascular system than a support for the vascular segment. In this case, the stent graft extends over the vascular segment to be replaced thus enabling reliable fixing of the stent graft to the vascular system of the patient. If the stent graft is embodied to replace a vascular segment and perform the function of a vascular prosthesis, it can in particular be unsuitable for minimally invasive surgery and only have a predetermined conformation.

According to a further aspect of at least one embodiment of the invention, the first hemodynamic parameter is determined based on a change in the vascular segment between the different time points. In this case, the change in the vascular segment is particularly suitable for the determination of a first hemodynamic parameter since a change of this kind interacts directly with the blood flow. The change can in particular relate to a geometric change.

According to a further aspect of at least one embodiment of the invention, the change relates to the expansion of the vascular segment. Modern imaging devices enable the expansion of a vascular segment to be determined precisely. In this case, the expansion can in particular relate to the diameter in a cross-sectional plane of the vascular segment. The expansion can furthermore relate to both the external diameter and the internal diameter of the vascular segment. A plurality of first hemodynamic parameters can be determined based on the change to the expansion of the vascular segment.

According to a further aspect of at least one embodiment of the invention, the maximum expansion of the vascular segment and the minimum expansion of the vascular segment are determined during at least one cardiac cycle of the patient. This enables a first hemodynamic parameter to be determined particularly precisely since the difference of the expansions between the maximum expansion and the minimum expansion is particularly great and hence provides particularly useful information on the change to the expansion of the vascular segment. The maximum expansion is typically measured during the systolic phase and the minimum expansion is typically measured during the diastolic phase.

According to a further aspect of at least one embodiment of the invention, the first hemodynamic parameter at least relates to the elasticity of the vascular segment or the blood flow rate in the vascular segment or the blood pressure in the vascular segment. These hemodynamic parameters are particularly important for the evaluation of the condition of a vascular segment. Therefore, the stent graft can be created such that one or more of these hemodynamic parameters lie within a predetermined value range during the use of the stent graft.

According to a further aspect of at least one embodiment of the invention, the first hemodynamic parameter relates to the elasticity of the vascular segment, wherein the stent graft is created from at least two different materials, wherein the materials are selected such that the stent graft has a first elasticity. In particular, the first elasticity can be calculated such that the stent graft together with the vascular segment and/or together with regions of the vascular system of the patient adjacent to the vascular segment has a specific elasticity. For the purposes of the present application, the elasticity can be a compressive modulus, a shear modulus or a modulus of elasticity. The elasticity can also be a predetermined value range around one of the moduli named. A predetermined modulus of this kind or the predetermined value range advantageously corresponds to a healthy patient.

The modulus of elasticity is also known as Young's modulus and is given in SI units von N/m² and hence has the unit of mechanical stress. The modulus of elasticity has a great influence on how the stent graft reacts to the pressure of a fluid flowing through the stent graft. The lower the modulus of elasticity of the stent graft, the greater the expansion of the stent graft at a specific pressure of the flowing fluid. The expansion of the stent graft is also accompanied by an increase in the diameter of the stent graft and hence the volume flow rate through the stent graft increases. For laminar flows, this relationship is expressed by the Hagen-Poiseuille law.

A specific elasticity is important for the transportation of blood through the vascular system of a patient. During systolic phase of the heart, the blood is pumped from the heart into the vascular system and the pressure in the vascular system rises. During the diastolic phase of the heart, the heart fills with blood and the pressure in the vascular system drops. Due to the pressure that builds up during the systolic phase and the associated expansion, the vascular system performs the function of an energy store. Due to the contraction of the blood vessels, the blood also flows through the vascular system during the diastolic phase. If the elasticity of a vascular segment is too low or too high, this vascular segment is no longer able to perform the function of an energy store in the envisaged manner. For example, with peripheral arterial occlusive disease, a pathologically changed elasticity of vascular segments results in a serious risk to the patient.

Furthermore, the elasticity can be anisotropic so that the elasticity of the stent graft has different values along different directions. Therefore, the first elasticity can also be calculated for only one value for one or more predetermined direction(s), in particular within the cross-sectional plane or perpendicularly to the central line. Alternatively, the first elasticity can be anisotropic and different values are calculated for different directions.

The different materials can in particular have different degrees of elasticity in cured condition. If the materials are mixed in a controlled manner, the resulting elasticity of the mixed materials can be controlled. Furthermore, during the creation of the stent graft, the different materials can be used such that the stent graft has an inhomogeneous or anisotropic material property, in particular an inhomogeneous or anisotropic elastic property. The materials can be plastics.

According to a further aspect of at least one embodiment of the invention, the first hemodynamic parameter is furthermore calculated based on a database, wherein a plurality of further hemodynamic parameters is stored in the database. This aspect of the invention enables the first hemodynamic parameter to be calculated taking into account additional information. A further hemodynamic parameter stored in the database can in particular correspond to a healthy vascular segment. A database of this kind can store a plurality of hemodynamic parameters, in particular as a function of further parameters such as a specific vascular segment or a specific patient group.

According to a further aspect of at least one embodiment of the invention, a central line of the vascular segment is determined on the basis of the first image data set, wherein the spatial course of the vascular segment is determined based on the central line. A central line of this kind can in particular be determined for the segmentation of the vascular segment or following the segmentation of the vascular segment. The spatial course of the vascular segment is determined particularly precisely if it is determined based on the central line. It is also possible for more than one central line to be determined when the vascular segment is branched.

According to a further aspect of at least one embodiment of the invention, the determination of the spatial course of the vascular segment entails the determination of the spatial course of the expansion along the central line. The expansion of a vascular segment can change along the central line. The course of the vascular segment is determined particularly precisely if the course and hence the change to the expansion along the central line are also determined. In this case, the expansion can in particular relate to the diameter in a cross-sectional plane of the vascular segment. The expansion can furthermore relate to both the external diameter and the internal diameter of the vascular segment. It is particularly important to determine the course and hence the change to the internal diameter of the vascular segment since this is the decisive factor for the determination of the flow properties. Hence, it is possible to determine whether the vascular segment is pathologically constricted, for example due to an abruptly reduced diameter.

The digital 3D model can comprise the structure and material properties of the stent graft. The structure also comprises the surface of the stent graft. In particular, the 3D model can indicate which material properties should be present at which region of the stent graft. Furthermore, the 3D model information can contain information as to which materials should be processed in which region of the stent graft and/or how these materials should be processed. For example, the digital 3D model can comprise information on the layer thickness of individual layers, which are to be applied by way of a (Poly)Jet method.

According to a further aspect of at least one embodiment of the invention, the digital 3D model is transferred via a network to the 3D printer. This enables the stent graft to be produced at different places. The digital 3D model can include all the information required for the creation of the stent graft. However, the 3D model can also be modified after transfer. For example, the digital 3D model can be provided in STL (abbreviation for surface tessellation language). In particular, the network can be an intranet or the internet.

Furthermore, at least one embodiment of the invention relates to a computing and printing unit for creating a stent graft, comprising the following units:

a first interface embodied to receive at least one first 3D image data set, wherein the first 3D image data set comprises 3D images of a vascular segment of a patient at different time points,

a determination unit embodied for the first determination of a first hemodynamic parameter of the vascular segment on the basis of the first image data set and the second determination of the spatial course of the vascular segment on the basis of the first 3D image data set,

a computing unit embodied to calculate a digital 3D model of the stent graft on the basis of the first hemodynamic parameter of the vascular segment and on the basis of the spatial course of the vascular segment,

a 3D printer embodied to create a stent graft on the basis of the digital 3D model.

A computing and printing unit of this kind can in particular be embodied to carry out the above-described method according to embodiments of the invention and its aspects. The computing and printing unit is embodied to carry out this method and its aspects in that the first interface, the determination unit, the computing unit and the 3D printer are embodied to carry out the corresponding method steps. At least one embodiment of the invention furthermore relates to an imaging device embodied to record the first 3D image data set comprising a computing and printing unit according to the invention.

The imaging device can be a tomography device, in particular a computed tomography device or a magnetic resonance imaging device. Furthermore, the imaging device can be an X-ray device, such as a C-arm X-ray device. The imaging device can in particular be an ultrasound device designed to record a 3D image data set. The first 3D image data set comprises a plurality of 3D images of the vascular segment at different time points. The 3D images at least depict the vascular segment for which a stent graft is to be created. The 3D images can in particular be tomographic images. For example, during the recording of a 3D image data set with a tomographic device at different time points, a plurality of measured data is acquired. This measured data can be used for the reconstruction of 3D images with different temporal foci. In this context, the reconstructed 3D images should be assigned to different time points. The measured data for the 3D image data set can in particular be acquired during an individual so-called scan.

For the purposes of the present application, “3D” designates a spatially three-dimensional property. Since the first 3D image data set comprises 3D images at different time points, the first 3D image data set can also be designated a 4D image data set. In this case, “4D” designates a spatially three-dimensional property and a temporal property.

At least one embodiment of the invention also relates to a computer program product with a computer program and a computer-readable medium. A substantially software-based implementation has the advantage that controller devices already used to date can be simply retrofitted with a software update in order to operate in accordance with at least one embodiment of the invention. In addition to the computer program, a computer program product of this kind can optionally comprise additional components such as, for example, documentation and/or additional components including hardware components, such as, for example, hardware keys (dongles etc.) for using the software.

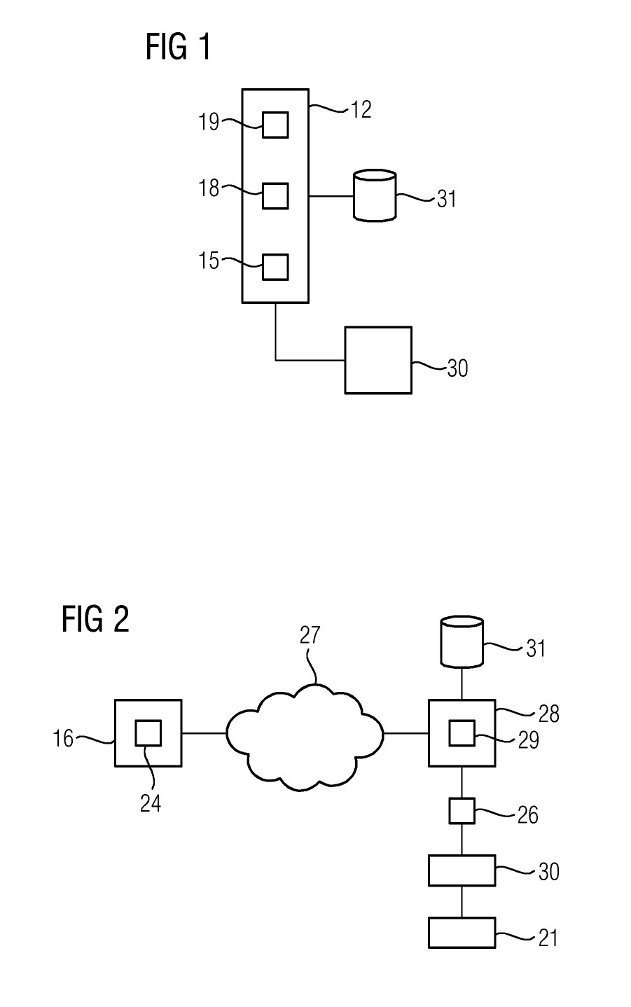

The computing and printing units shown here and the imaging device shown here are designed to carry out a method according to the invention. FIG. 1 shows a computing and printing unit. The computing and printing unit shown here comprises a first interface 19, a determination unit 18, a computing unit 15 and a 3D printer 30. The first interface 19 can entail generally known hardware or software interfaces, for example the hardware-interfaces PCI-Bus, USB or Firewire. Both the determination unit 18 and the computing unit 15 can comprise software elements and hardware elements, for example a microprocessor or a so-called FPGA (abbreviation for “field programmable gate array”). The determination unit 18 and the computing unit 15 can be part of a computer 12. Furthermore, the computing and printing units are able to communicate with a database 31. The computing and printing unit can further comprise interfaces, in particular for communication with the database 31 and the 3D printer 30.

FIG. 2 shows a network with a computing and printing unit. The first 3D image data set 24 is stored on a server 16 and can be transferred via a network 27 to the client 28. In the embodiment shown here, this client 28 comprises the interface 19, the determination unit 18, and the computing unit 15. A computer program 29 according to the invention is stored on the client 28 in an executable way. The client 28 has access to a database 31 on which a plurality of hemodynamic parameters is stored. In the example embodiment shown here, the calculated 3D model 26 is transferred directly to a 3D printer 30. In a further example, not shown here, the 3D model 26 is transferred back to the server 16 or to another client. Here, the transfer TRF of the digital 3D model to the 3D printer 30 can also take place via a network 27. Accordingly, the 3D printer 30 can also be connected to the server 16 or another client.

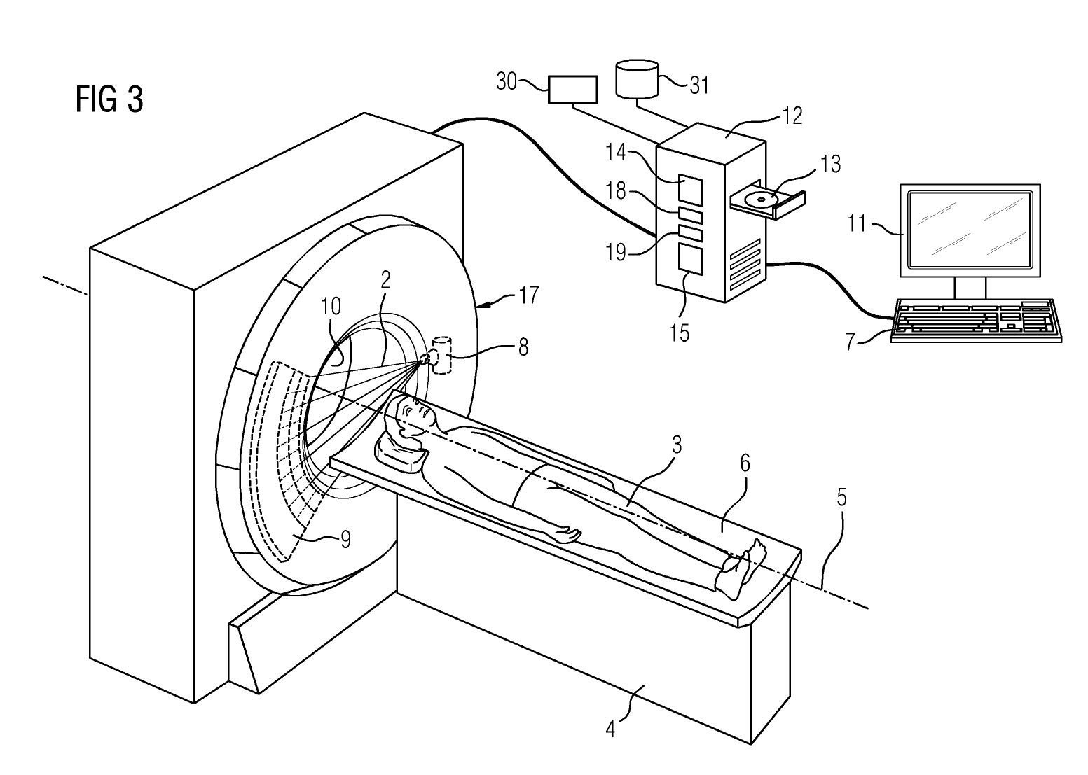

FIG. 3 shows an imaging device using example of a computed tomography device. The computed tomography device shown here has a recording unit 17 comprising an X-ray source 8 and an X-ray detector 9. During the acquisition of measured data, the recording unit 17 rotates about a system axis 5 and, during the recording, the X-ray source 8 emits X-rays 2. In the example shown here, the X-ray source 8 is an X-ray tube. In the example shown here, the X-ray detector 9 is a line detector with a plurality of lines.

In the example shown here, a patient 3 lies on a patient bed 6 during the acquisition of measured data. The patient bed 6 is connected to a bed base 4 such that the base supports the patient bed 6 with the patient 3. The patient bed 6 is designed to move the patient 3 along a receiving direction through the opening 10 of the recording unit 17. The receiving direction is as a rule defined by the system axis 5 about which the recording unit 17 rotates during the recording of measured data. In the case of a spiral recording, the patient bed 6 is moved continuously through the opening 10 while the recording unit 17 rotates about the patient 3 and acquires measured data. Thus the X-rays 2 describe a spiral on the surface of the patient 3. For the reconstruction of 3D images 25 based on the measured data, the computed tomography device shown here furthermore has a reconstruction unit 14.

In addition, an imaging device such as the computed tomography device shown here can also have a contrast agent injector for the injection of contrast agent into the blood circulation of the patient 3. This enables the 3D images 25 to be recorded using a contrast agent such that the vascular segment 20 can be depicted with increased contrast. Furthermore, the contrast agent injector also provides the possibility of actuating angiographic recordings or carrying out perfusion scanning. Contrast agents should generally be understood to be means which improve the depiction of body structures and functions during imaging methods. In the context of the present application, contrast agents should be understood to mean both conventional contrast agents such as, for example, iodine or gadolinium and tracers such as, for example, 18F, 11C, 15O or 13N.

In the example shown here, the first interface 19 is embodied as part of the computer 12. The computer 12 is connected to an output unit in the form of a screen 11 and an input unit 7. The 3D images 25 can be depicted in different forms on the screen, for example as rendered volume images or as sectional views. The input unit 7 is, for example, a keyboard, a mouse, a so-called “touch screen” or even a microphone for voice input. The input unit 7 can be used to start a computer program 29 according to the invention. The individual steps of the method according to the invention can be supported by the input unit 7; for example, a click of a mouse can confirm a selection of a vascular segment in a 3D image.

The computed tomography device shown here comprises a reconstruction unit 14 for the reconstruction of 3D images. Furthermore, the computer 12 comprises a determination unit 18 and a computing unit 15. The computer 12 and the units assigned thereto can interact with a computer-readable medium 13, in particular in order to carry out a method according to an embodiment of the invention via a computer program 29 with a program code. Furthermore, the program code of the computer program 29 can be stored in a retrievable way on the machine-readable medium 13. In particular, a machine-readable medium can be a CD, DVD, Blu-Ray disk, a memory stick or a hard disk. In this case, the computer program product comprises the computer program 29 and the corresponding program code.

In the embodiment shown here, at least one computer program 29 is stored on the memory of the computer 12, which carries out all the method steps of the method according to the invention when the computer program 29 is executed on the computing unit 15. The computer program 29 for carrying out the method steps of the method according to the invention comprises a program code. Furthermore, the computer program 29 can be embodied as an executable file and/or on a computing system other than the computer 12. For example, the computing and printing unit can be designed such that the computer program 29 is loaded into the memory of the computing and printing unit for carrying out the method according to the invention via an intranet or via the internet.

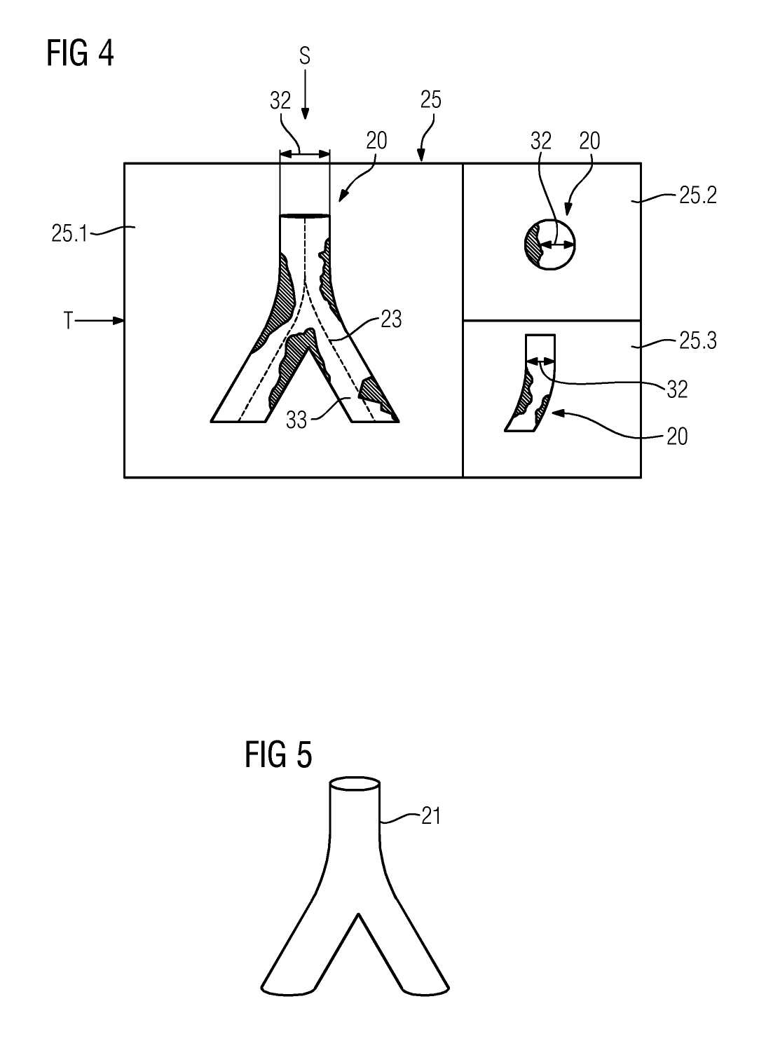

FIG. 4 shows a 3D image of a vascular segment. In the example shown here, the vascular segment 20 is a part of the aorta in the abdominal cavity. Here, the 3D image 25 is depicted in the form of three different sectional views. The first sectional view 25.1 corresponds to the frontal plane, the second sectional view 25.2 corresponds to the transversal plane and the third sectional view 25.3 corresponds to the sagittal plane. The arrow designated T indicates the position of the second sectional view 25.2 and the arrow designated S indicates the position of the third sectional view 25.3. In the first sectional view 25.1, the central line 23 of the vascular segment 20 is also depicted as a dashed line. Furthermore, the change to the expansion of the vascular segment 20 along the central line 12 is shown. Here, the expansion is indicated at different positions by way of example by the internal diameter 32 of the vascular segment 20. In the example shown here, there is a stenosis 33, i.e. a narrowing of the vascular segment. The vascular segment 20 shown here is an important blood-carrying vessel so that it is desirable to treat the stenosis 33 such that the hemodynamic properties of the vascular segment 20 are adapted by a stent graft 21 such that they conform to those of a healthy patient 3. Therefore, the stent graft 21 is intended, on the one hand, to support the vascular segment 20 and, on the other, to influence the hemodynamic properties. The invention described here enables a stent graft 21 to be depicted such that it is adapted individually to the anatomy of the patient 3 and in this way improves the function of the vascular segment 20.

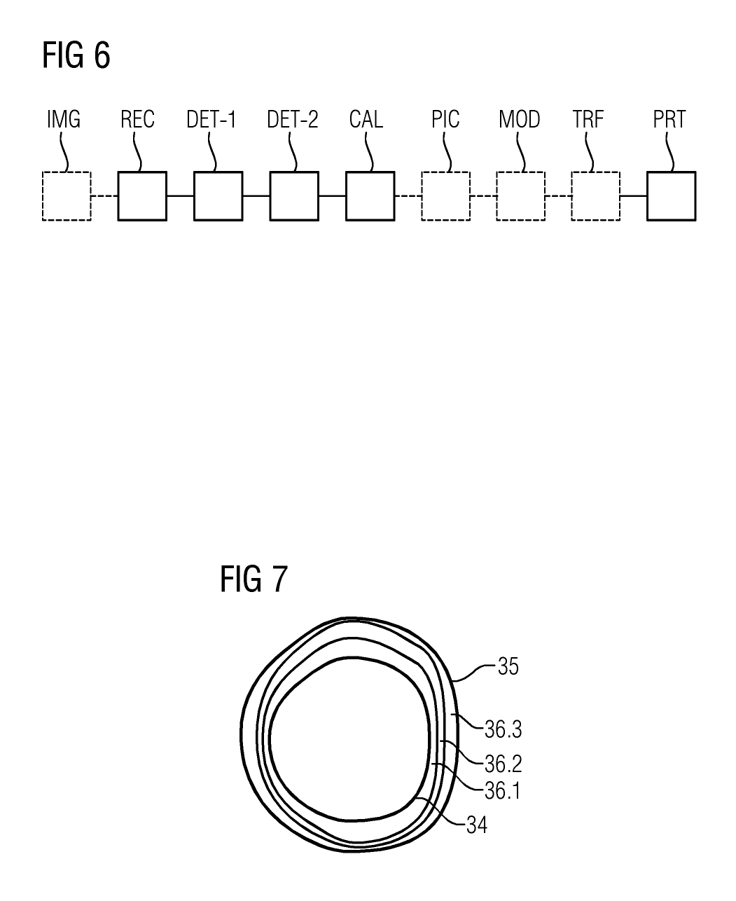

FIG. 5 shows a stent graft. This stent graft is in particular suitable to support or replace the vascular segment 20 shown in FIG. 4. A stent graft 21 of this kind according to the invention can be produced with the method illustrated in FIG. 6. This method can optionally also comprise the imaging IMG of a first 3D image data set 24. In this case, the imaging IMG also comprises the reconstruction of the 3D images 25. The stent graft 21 is based on the reception REC of the first 3D image data set 24 via a interface 19, wherein the first 3D image data set comprises 3D images 25 of a vascular segment 20 of a patient 3 at different time points. There is now a first determination DET-1 of a first hemodynamic parameter of the vascular segment on the basis of the first 3D image data set 24 via a determination unit 18 and a second determination DET-2 of the spatial course of the vascular segment 20 on the basis of the first 3D image data set 24 via the determination unit 18.

The first hemodynamic parameter can in particular be the elasticity of the vascular segment 20, the blood flow rate in the vascular segment 20 or the blood pressure in the vascular segment 20. The elasticity can be calculated directly from a change to the expansion, in particular the diameter, of the vascular segment 20. In this case Hooke's law can be used in a simple approximation. It is also possible to determine the elasticity via a simulation. The blood flow rate can be determined by way of ultrasound. New methods also permit the measurement of the blood flow rate via magnetic resonance imaging, phase contrast angiography and computed tomography. Here, reference is made by way of example to the German patent application with the filing reference 102015205959.6, the entire contents of which are hereby incorporated herein by reference.

According to a further embodiment of the invention, a second image data set is received by the interface 19, wherein the first determination DET-1 of a first hemodynamic parameter of the vascular segment is performed on the basis of the second image data set of the vascular segment 20 via the determination unit 18. The embodiment of the invention can also comprise the recording of the second image data set. Furthermore, the first 3D image data set 25 can comprise the second image data set. For example, the first 3D image data set 24 comprises a plurality of tomographic images and the second image data set comprises ultrasound images. It is now possible to determine the blood flow rate based on the ultrasound images. Furthermore, the second image data set can be an elastographic image data set. Elastography is based on ultrasound or magnetic resonance imaging and enables the elasticity of the vascular segment 20 to be determined.

The blood pressure in the vascular segment 20 can in particular be determined very precisely by way of a flow simulation. A further hemodynamic parameter determine can be the FFR (abbreviation for fractional flow reserve), wherein the FFR indicates the drop in pressure due to a stenosis 33. Therefore, the stent graft 21 can be set such that the FFR for the vascular segment 20 with a stenosis 33 falls below a predetermined value due to the stent graft 21.

This is followed by the calculation CAL of a digital 3D model 26 of the stent graft 21 on the basis of the first hemodynamic parameter of the vascular segment 20 and on the basis of the spatial course of the vascular segment 20 via a computing unit 15. This 3D model 26 can in particular define properties of the stent graft 21 such as its material composition, the thickness of the walls of the stent graft 21 etc. The 3D model 26 can be calculated based on the segmented structure of the vascular segment 20 in at least one the 3D images 25. The segmentation can be performed by way of usual algorithms such as a region-oriented algorithm or an edge-oriented algorithm. It is also possible to take account of predetermined limiting conditions during the calculation CAL.

Optionally, it is also possible to carry out a step for the modification MOD of the 3D model 26. The modification MOD can be performed based on the input of a user of the computing and printing unit or be based on predetermined limiting conditions. The input can cause the direct modification MOD of the 3D model 26, for example in that the thickness of the walls of the stent graft 21 or the diameter of the stent graft 21 are set. For example, a limiting condition can relate to the stability of the stent graft 21 or the thickness of the walls of the stent graft 21. It is also possible for a user to input a limiting condition. Furthermore, optionally the step PIC can be performed to display the 3D model 26. For example, the 3D model 26 can be displayed on a display unit 11. The modification MOD can be performed by an interaction of the user via an input unit 11 and the display unit 11 with the displayed model 26. For example, a region of the displayed model 26 can be marked and the marked region 26 displaced. Furthermore, the modification MOD can include scaling of the 3D model.

The 3D model 26 can be available in different formats or be converted into different formats. In particular, the 3D model 26 can be in STL format. The 3D model 26 is now transferred to the 3D printer 30, for example by way of a transfer TRF via a network 27. This is followed by the creation PRT of a stent graft 21 on the basis of the digital 3D model 26 via a 3D printer 30. In this case, the 3D printer 30 converts the information on the structure, material composition etc. from the 3D model 26 into a stent graft 21 by way of printing process.

The stent graft 20 can be printed such that it has a smooth surface. Furthermore, it can be printed such that, apart from the inputs and outputs for a flowing fluid, that it has an enclosed surface. Alternatively, the stent graft 20 can be printed such that it has a grid-shaped or net-shaped structure. In further embodiments of the invention, the term “based” can be replaced by “as a function of” or “in functional dependence on”.

FIG. 7 shows a cross section through a stent graft. Hence, the stent graft 20 also extends into the image plane and out of the plane. The cross section is bounded in the image plane by an internal contour 34 and by an external contour 36. This example shown shows a stent graft 20 comprising a plurality of different materials. In the example shown here, three different materials are used but it is also possible to use more or fewer different materials. In the example shown here, the first material 36.1 has the lowest modulus of elasticity of the materials used. The third material 36.3 has the greatest modulus of elasticity auf. The modulus of elasticity of the second material 36.2 lies between that of the two other materials. The materials are arranged in layers. In the example shown here, the layers have different thicknesses. In other embodiments, the layers can also have the same thicknesses. Furthermore, the layers can be arranged concentrically.

The embodiment shown here has the advantage that the elastic, soft inner side of the stent graft 20 is particularly suitable for yielding to the pressure of a fluid flowing in the stent graft 20 and hence for supporting the propagation of waves of the flowing fluid. A less elastic, hard outer side of the stent graft 20 favors a constant external shape of the stent graft so that said stent graft is stable.

The aforementioned description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure and appended claims.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one embodiment of the invention relates to a non-transitory computer-readable storage medium comprising electronically readable control information stored thereon, configured in such that when the storage medium is used in a controller of a magnetic resonance device, at least one embodiment of the method is carried out.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for creating a stent graft, comprising:
receiving at least one first 3D image data set via a first interface, wherein the 3D image data set includes 3D images of a vascular segment of a patient at different time points;
determining a first hemodynamic parameter of the vascular segment, based at least on the first 3D image data set, via a determination unit;
determining the spatial course of the vascular segment, based at least on the first 3D image data set, via the determination unit;
calculating a digital 3D model of the stent graft, on the basis of the first hemodynamic parameter of the vascular segment and on the basis of the spatial course of the vascular segment, via a computing unit; and
creating the stent graft, on the basis of the digital 3D model, via a 3D printer.

2. The method of claim 1, wherein the first hemodynamic parameter is determined based on a change in the vascular segment between the different time points.

3. The method of claim 2, wherein the change relates to expansion of the vascular segment.

4. The method of claim 3, wherein a maximum expansion of the vascular segment and a minimum expansion of the vascular segment are determined during at least one cardiac cycle of the patient.

5. The method of claim 1, wherein the first hemodynamic parameter relates to at least one of the following parameters:
the elasticity of the vascular segment,
the blood flow rate in the vascular segment, and
the blood pressure in the vascular segment.

6. The method of claim 1, wherein the first hemodynamic parameter relates to the elasticity of the vascular segment, wherein the stent graft is created via at least two different materials, and wherein the materials are selected such that the stent graft has a first elasticity.

7. The method of claim 1, wherein the first hemodynamic parameter is furthermore calculated based on a database, and wherein a plurality of further hemodynamic parameters is stored in the database.

8. The method of claim 1, wherein a central line of the vascular segment is determined on the basis of the first 3D image data set, and wherein the spatial course of the vascular segment is determined based on the central line.

9. The method of claim 1, wherein the determination of the spatial course of the vascular segment includes a determination of the spatial course of the expansion along the central line.

10. The method of claim 1, furthermore comprising:
transferring the digital 3D model to the 3D printer via a network.

11. A computing and printing unit for creating a stent graft, comprising:
a first interface embodied to receive at least one first 3D image data set, wherein the first 3D image data set includes 3D images of a vascular segment of a patient at different time points;
a determination unit embodied for the first determination of a first hemodynamic parameter of the vascular segment on the basis of the first 3D image data set and the second determination of the spatial course of the vascular segment on the basis of the first 3D image data set;
computing unit embodied to calculate a digital 3D model of the stent graft on the basis of the first hemodynamic parameter of the vascular segment and on the basis of the spatial course of the vascular segment; and
3D printer embodied to create a stent graft on the basis of the digital 3D model.

12. The computing and printing unit of claim 11, embodied to carry out at least:
receiving at least one first 3D image data set via the first interface, wherein the 3D image data set includes 3D images of a vascular segment of a patient at different time points;
determining a first hemodynamic parameter of the vascular segment, based at least on the first 3D image data set, via the determination unit;
determining the spatial course of the vascular segment, based at least on the first 3D image data set, via the determination unit;
calculating a digital 3D model of the stent graft, on the basis of the first hemodynamic parameter of the vascular segment and on the basis of the spatial course of the vascular segment, via the computing unit; and
creating the stent graft, on the basis of the digital 3D model, via the 3D printer.

13. An imaging device embodied to record the first 3D image data set comprising:
the computing and printing unit of claim 11.

14. A non-transitory computer program product including a computer program, loadable directly into a memory of computing and printing unit, with program segments for carrying out the method of claim 1 when the program segments are executed by the computing and printing unit.

15. A non-transitory computer-readable medium including program segments, readable and executable by a computing and printing unit, to carry out the method of claim 1 when the program segments are executed by the computing and printing unit.

16. A stent graft produced by the method of claim 1.

17. An imaging device embodied to record the first 3D image data set comprising:
the computing and printing unit of claim 12.

18. A non-transitory computer program product including a computer program, loadable directly into a memory of computing and printing unit, with program segments for carrying out the method of claim 2 when the program segments are executed by the computing and printing unit.

19. A non-transitory computer-readable medium including program segments, readable and executable by a computing and printing unit, to carry out the method of claim 2 when the program segments are executed by the computing and printing unit.

20. A stent graft produced by the method of claim 2.
